(19) United States
(12) Patent Application Publication
(10) Pub. No.: US 2005/0096852 A1
(43) Pub. Date: May 5, 2005

(54) Method for increasing the stability and intensity of odorant molecules

(76) Inventor: Luca Turin, London (GB)

Correspondence Address: Womble Carlyle Sandridge & Rice, P.O. Box 7037, Atlanta, GA, 30357-0037, US

(21) Appl. No.: 10/868,489

(22) Filed: Jun. 15, 2004

Related U.S. Application Data
(63) Continuation of application No. PCT/US02/26438, filed on Aug. 20, 2002.
(60) Provisional application No. 60/377,914, filed on May 3, 2002.
(60) Provisional application No. 60/389,298, filed on Jun. 17, 2002.
(60) Provisional application No. 60/355,052, filed on Feb. 7, 2002.
(60) Provisional application No. 60/342,150, filed on Dec. 19, 2001.
(60) Provisional application No. 60/348,580, filed on Jan. 15, 2002.

(51) Int. Cl. G06F 19/00; G01N 31/00; A61K 7/00

(52) U.S. Cl. 702/22; 424/401

(57) ABSTRACT

Methods for determining determinants of an odorant, such as character, intensity and stability, are described. Odorant character is determined based on the vibrational spectra of the odorant, odor intensity is determined based on zinc-binding affinity, and stability is based on an analysis of the odorant in a particular environment. The methods described herein permit one to design novel odorant molecules with increased zinc-binding ability, with all other things such as size volatility and hydrophobicity being equal, to increase the intensity of an odorant derivative relative to a parent odorant from which it is derived. Combinatorial libraries of odorants (“odotopic libraries”) to be evaluated for odor character and intensity are also disclosed, as are high throughput methods of evaluating these compounds. Further, methods for identifying an odorant with similar odor character but with improved intensity, stability or other improved physical and/or chemical properties are disclosed.

### FIELD OF THE INVENTION

The present invention relates generally to the field of flavorings and fragrances. More particularly, the present invention relates to methods of identifying derivatives of aromachemicals with improved physical and/or chemical properties over the parent aromachemicals, for example, increased stability and/or increased odor intensity. This application is a continuation of PCT/US02/26438, filed 20 Aug. 2002; which claims priority to U.S. Provisional Application No. 60/377,914, filed May 3, 2002; No. 60/389,298, filed Jun. 17, 2002; No. 60/355,052, filed Feb. 7, 2002; No. 60/342,150, filed Dec. 19, 2001, NO. 60/348,580, filed Jan. 15, 2002, the contents of each of which are hereby incorporated by reference.

### BACKGROUND OF THE INVENTION

There is no clear understanding yet of the relationship between a molecule's structure and its odor character (smell type) and intensity (potency). This is a biological question that requires an understanding of the receptor recognition mechanism to answer. The presently preferred theory is that molecular shape determines smell. This is a lock and key-type approach to smell recognition, but this approach has failed. Because there is currently no universal theory regarding smell recognition, research in the field accordingly proceeds by a very expensive and time-consuming approach of trial and error.

The present inventor recently proposed an alternative theory that molecular vibrations of the odorant determine its smell. The theory helps explain certain hitherto mysterious features of human olfaction., e.g. our ability to smell functional groups such as SH, CN, etc. This theory posits that the receptors function as solid-state spectroscopes, using inelastic electron tunneling to detect molecular vibrations. Using this theory, it should be possible to calculate the vibrational spectrum of an odorant as seen by a tunneling spectroscope. Vibrational modes (frequencies) of odorants are easily calculated.

The present inventor has developed a method for calculating intensities of these modes, and for allowing for the finite frequency resolution of the system. He has further developed a method for quantitatively comparing spectra, and for mapping the results in “odor space.” The method has been used to predict the smell of novel odorants, and in particular to determine which alterations to a given odorant's structure might alter its chemical properties in a desirable fashion without altering its smell. However, a limitation of this method is that it does not predict an odorant's intensity, only its character.

A peculiar feature of a spectroscopic theory of olfaction is that it requires odor character and intensity to be considered separately. Common odorants differ by several orders of magnitude in their intensity, despite the fact that their spectra will generally be of comparable magnitude. Thus, it is theorized that the main determinant of odor intensity is affinity of the molecule for the receptor.

It would be advantageous to have a method for separately predicting the two basic determinants of an odorant, namely character and intensity. The present invention provides such a method.

### SUMMARY OF THE INVENTION

Methods for determining determinants of an odorant, such as character, intensity and stability, are described. Odorant character is determined based on the vibrational spectra of the odorant, odor intensity is determined based on zinc-binding affinity, and stability is based on an analysis of the odorant in a particular environment.

Accordingly, the methods described herein permit one to design novel odorant molecules with increased zinc-binding ability, with all other things such as size volatility and hydrophobicity being equal, to increase the intensity of an odorant derivative relative to a parent odorant from which it is derived. That is to say, if a known odorant has a desirable odor but it would benefit from an increased odor intensity, the methods described herein permit one to design an odorant with similar or substantially identical odor but with increased zinc-binding affinity, and therefore, increased odor intensity. Similarly, if a known odorant has a desirable odor but it would benefit from an increased stability in a particular environment, the methods described herein permit one to design an odorant with similar or substantially identical odor but with increased stability in a particular environment.

Combinatorial libraries of odorants (“odotopic libraries”) to be evaluated for odor character and intensity are also disclosed, as are high throughput methods of evaluating these compounds. Further, methods for identifying an odorant with similar odor character but with improved intensity, stability or other improved physical and/or chemical properties are disclosed.

### BRIEF DESCRIPTION OF THE DRAWINGS

FIGS. 1A-F represent graphs of the vibrational spectra of various cyclopropyl analogues of citral and an analogue of citral where the aldehyde group has been replaced with a nitrile group.

FIGS. 2A-D represent graphs of the vibrational spectra of rose oxide, linalool, limonene and ionone with CP, OX and TH substitutions.

FIG. 3 represents a graph of the vibrational spectra of lilial® and a derivative of lilial® in which the phenyl ring is replaced with a thiophene ring.

### DETAILED DESCRIPTION OF THE INVENTION

The understanding of what causes odor determination enhances the ability to rationally design odorants. This understanding, coupled with an understanding of what causes odorant stability or instability and/or what causes odorant intensity, further enhances the ability to rationally design odorants. The present methods enable one to rationally design odorants with improved physical and chemical properties, or, alternatively, to use combinatorial chemistry (irrational odorant design) to identify odorants with desirable physical and/or chemical properties.

The methods are based on the understanding that compounds with similar vibrational spectra have similar odorant character. Further, by understanding what substitutions are possible, one can select an replacement that provides desired physical and/or chemical properties, such as improved stability in a particular environment and/or improved odor intensity.

The methods can be used to develop and evaluate combinatorial libraries of odorants. This allows for rapid identification of suitable odotopic replacements for known odorants, wherein the replacements have improved properties over the parent odorants.

Improved Binding of Aromachemicals for the Receptors

One hypothesis for how odorants bind to the receptor involves metal binding. A metal ion is bound to the receptor with some coordination sites left free, and the molecule binds to the coordination site and therefore to the receptor protein. In theory, one can increase the affinity of hydrophobic molecules to their receptors by improving the binding of the molecules to the metal ions.

While not wishing to be bound to a particular theory, it is believed that odor intensity is due, at least in part, to the affinity of molecules for their receptors. The affinity of molecules for their receptors depends, at least in part, on the ability of odorants to bind to a metal ion contained in the receptor protein, which is, at least in some embodiments, a zinc ion.

The nature of the odorant binding site within olfactory receptors was raised in Turin, Chemical Senses, 21(6):773-791 (1996) and it is believed to be close to or coincident with a previously unreported and highly conserved zinc-binding sequence in olfactory receptors. This tallies with known effects of zinc, or lack of it, on olfaction. It also tallies with the fact that many of the strongest odorants can bind metals. This correlation helps explain how stereoisomers differing in intensity also differ in their ability to bind zinc. While hydrophobicity and the presence of large partial charges on atoms play a role in odor intensity, it is believed that one of the main determinants of intensity is zinc-binding. The ability of an odorant to bind zinc is predictable, even in a quantitative fashion, using quantum chemistry software.

The Calculation of Odor Character

Similarity in vibrational spectra correlates rather well with similarity in odor character. By analogy with color vision, the spectral data is probably analyzed by the brain in discrete spectral bands corresponding to discrete receptor families. Their position, width and number is unknown. As a result, it is not yet possible to map spectra into odor space such that it perfectly correlates with how odor is detected in nature. However, what this method gives is a metric of the odor space such that close spectra are likely to give close smells. Note that similarity of smell does not guarantee similarity of naming. Much in the way that some countries on a map may be smaller or bigger, some odor space territories may be small, others large. For example, lemon is unquestionably close to both rose and lime, because many compounds exist whose odor character uses these descriptors.

Inelastic electron tunneling spectroscopy (IETS) theory is well known to those of skill in the art (see, for example, Turin, Chemical Senses, “A Spectroscopic Mechanism for Primary Olefactory Reception,” 21(6):773-791 (1996)). This theory can be used to calculate the vibrational spectra of molecules as perceived by olfactory receptors. A molecule composed of N atoms has 3N-6 vibrational modes. Each measured molecular vibration is described by three parameters: frequency, intensity and width. Vibrational frequencies can be calculated with reasonable accuracy using quantum chemistry software. Line width, i.e. the resolution of the system, is unlikely to better than kT. Line intensity can be estimated using existing IETS theory. The result of these calculations is a spectrum. Alternatively, the spectra can be obtained using an appropriate spectrometer.

Compounds that possess similar tunneling vibrational spectra possess similar odorant character. Accordingly, the methods for predicting odorant character involve:
a) using any available methodology to calculate vibrational frequencies and atom displacements of a putative molecule (i.e., calculating frequency and movement),
b) using any available methodology to calculate electrostatic charge distribution on the constituent atoms of the putative molecule, and
c) calculating the intensity of each vibrational mode using the information in steps a and b and an algorithm that calculates intensity of vibrational modes as detected or perceived by a solid state electron tunneling spectrometer.

This information can be “smoothed” into the form of a spectra using known smoothing techniques. Basically, these techniques involve convolving each peak with an error function of a given width and shape and taking the sum of broadened peaks to generate a spectrum.

For any putative molecule, one can further calculate the same data on a number of putative analogs of the molecule to generate a virtual combinatorial library. This data can be mapped to an “odorant space” by:
a) breaking all of the spectra up into “n” fragments,
b) scaling the peaks of one compound relative to another compound such that at least one peak is the same height as another,
c) taking the remaining “n−1” peaks and generating a plot with the coordinates of those peaks, and
d) optionally, comparing the location on the odorant map of the various compounds.

Compounds with similar location in the odorant map will have similar odorant character. Ideally, to plot the data in a three dimensional space, using x, y, and z coordinates, n is 4 and (n−1) is three, representing the three portions of the spectra whose amplitude can be mapped in x, y and z coordinates.

There are several algorithms for calculating the spectra for odorants. Inefficient algorithms and slower computers, such as were available even a few years ago, would require a significant amount of time to calculate these spectra. However, with today's computer power, and more efficient algorithms, the spectra can be readily calculated. The actual spectra themselves, as well as the identify of the compounds whose spectra is calculated, take up very little storage space. Accordingly, existing technology (commercially available Macintosh IMAC computers and an appropriate algorithm) can generate odorant mapping coordinates of thousands of compounds in a relatively short time frame (i.e., in less than one week).

Neural networks can be trained to recognize compounds with an appropriate location on the odorant map such that they would be expected to possess similar odorant character. Relational databases can be used to identify compounds that have similar odorant character. Suitable relational databases are well known to those of skill in the art of combinatorial chemistry.

Molecular mechanics software is commercially available (TRIPOS, St. Louis, Mo.) for computing molecular energy, vibrational spectra, and various other properties of components of virtual combinatorial libraries. This software can easily be adapted to calculate the inelastic electron tunneling spectra (IETS) of virtual compound libraries using known algorithms.

Commercially available software also exists for storing and searching chemical structures and property data directly inside relational databases. It is now routine in the art to use relational databases, for example, Oracle® databases, to store data related to individual compounds in combinatorial libraries. Computer programs are also commercially available for use in developing molecular descriptors, clustering compounds, selecting diverse subsets, filtering compound lists by various physical and/or chemical properties, and comparing the diversity of compound databases. Complex virtual combinatorial libraries can be prepared using this type of software. Existing software is already being used in the pharmaceutical industry to rapidly determine structure-activity relationships, and can easily be adapted to rapidly determine structure-odor relationships using the technology described herein.

The in silico compound libraries, and relational databases including the IETS spectral data of the compounds in the in silico libraries, enable one to avoid costly synthesis and testing of chemical compounds that are unlikely to have desired odorant properties, and focus on those compounds that are most likely to have desired odorant properties.

In one embodiment, compounds that have subtle nuances of certain odors are identified by comparison to historical data stored on relational databases and/or using neural networks.

Using this technology, one can create a virtual combinatorial library of putative odorants and map the coordinates in “odorant space.” In one embodiment, a virtual three-dimensional space is created, where compounds are located as points in three dimensions. Once an ideal odor quadrant is identified, compounds located in the same virtual three dimensional space can be identified as potential “hits” for further screening. Such screening can involve actual chemical synthesis and evaluation.

The library can be assayed by comparing the odor character of a putative odorant with putative analogs to identify compounds that in theory would have the same or similar odorant character. These analogs can then be synthesized and the odorant character verified.

Similarly, one can create an actual odorant library of compounds which in theory would possess similar or identical odorant character, and screen the library for odorant character as well as other desired properties. Such properties include stability under certain conditions, odorant intensity, log P values, and the like. Some of these physical and/or chemical properties can be calculated, whereas some other properties can only be determined experimentally. Those of skill in the art can readily determine the log P values or stability of a compound in a given environment using conventional chemistry.

It can be much easier and less expensive to generate in silico libraries of thousands of molecules than to generate similar chemical libraries of compounds. When seeking to identify odorants with similar odorant character to an existing odorant, lead generation can be performed by calculating the spectra of the odorant, and then plotting the spectra in odorant space. The position in odorant space can be compared to a virtual, in silico library of compounds, and lead compounds identified. Lead optimization can be performed in silico by comparing the degree of overlap between the putative odotope and the odorant. Alternatively, once the in silico library is screened for potential leads, the leads can be chemically generated and compared with the odorant. Further, the leads can be compared on the basis of additional properties, such as odorant intensity, stability under certain physical/chemical environments, log P values and the like.

Odorant intensity can be calculated by measuring zinc binding, either theoretically, by calculating zinc-binding energies using computational chemistry methods, especially ab initio calculations, or experimentally, as described in Rakow N A, Suslick K S., “A calorimetric sensor array for odor visualization,” Nature 406(6797):710-3 (Aug. 17, 2000). This type of assay can be conducted in a high throughput manner, for example, on the compounds identified from the combinatorial library.

In determining an appropriate odotopic replacement for an odorant, the vibrational spectra can be calculated either experimentally by direct measurement, or using quantum theory. When two compounds spectra which overlap in an amount of 85% or more, these compounds have essentially the same odor character. As discussed in more detail below, many odotopic replacements are available to the odorant chemist, any and all of which can be used in connection with appropriate molecules to design improved odorants.

Making Better Odorants

The ability to calculate the effect of changes in odorant structure on odor character can be applied to an interesting and potentially important problem in fragrance chemistry, that of making improved versions of known odorants. The improvement can take several forms, but two stand out: greater intensity and greater chemical stability without change in odor character. If greater intensity is desired, then the odorant structure is modified in order to increase the intensity of the odor, such as by increasing zinc-binding ability, without significantly changing odor character. If greater stability is desired, then one or more structural features responsible for chemical instability are altered without significantly changing odor character.

The number of changes that can be made to a given odorant can be very large, for example, on the order of several thousand, considering removal, addition and/or replacement of even a fraction of the odorant component atoms. However, combinatorial chemistry can provide for rapid preparation and screening of odotopic replacements (“isodonic molecules”) for known odorants.

Isodonic Molecules

Certain odorant substructures that can be modified to result in odorant derivatives are correctly predicted by vibrational calculations, but it could be argued that they also preserve molecular shape. The neologism isodonic (neologism, from the Greek, vibration) is proposed to denote such molecules.

There are certain odorant substructures which can be interchanged without significantly effecting odor character. Isodonic replacements include, without limitation, aldehyde-nitrile replacement, aldehyde-methyl ether replacement, aldehyde-acetal replacement, ene-cyclopropane replacement, ene-oxirane replacement, ene-thiirane replacement, ene-thioether replacement, isobutenyl-phenyl replacement, isobutenyl-dimethyl cyclopropyl replacement, phenyl-dimethyl cyclopropyl replacement and benzene-thiophene replacement. These replacements are discussed in more detail below.

Double-bond Replacements and their Effect on Odor

Many aromachemicals include an isoprenyl unit and/or other C═C double bonds. The C═C double bonds can be replaced by thioether groups, —S—, without marked change in odor character. Alternatively, the carbon double bond can be replaced with a thiirane ring. In these embodiments, the lone pair of electrons on sulfur binds readily to Zn, which increases the odor intensity without significantly altering the odor type.

One or more (if present) C═C double bonds in an aromachemical can be replaced with cyclopropane, oxirane (OX), or thiirane (TH) moieties. The odor of the compounds remains substantially the same with this substitution, whereas the odorant intensity can be dramatically improved. The odorant intensity can be determined by measuring zinc binding affinity, for example, using the techniques described in Rakow N A, Suslick K S. A colorimetric sensor array for odor visualization. Nature. 2000 Aug. 17;406(6797):710-3., although other methods are available. All four 3-membered rings described above can coordinate to a zinc ion, but they do so in two different fashions. The cyclopropyl group coordinates via its C—C bonds which, because of the strained geometry, have a partial pi character. The three heterocycles coordinate by more conventional lone pair bonding. The 3-membered rings are shown here coordinated to a zinc ion in turn bound to an imidazole ring, which represents the histidine known to be involved in biological binding of zinc to olfactory receptors. The binding energies, as calculated by both semiempirical and ab initio methods are in the following order: TH>CP>DB>OX, wherein DB is the C═C double bond. Based on the binding energies, it is expected that thiirane and cyclopropane derivatives will bind better to zinc than the C═C double bond. However, there are other considerations, of course, which enter into the overall affinity of the odorant for its receptor, including but not limited to hydrophobicity, steric hindrance, and the like. The thiirane derivative appears to be the most stable derivative chemically.

Another advantage of replacing the C═C double bond with an oxirane or thiirane is that this produces a molecule with a higher molecular weight. The greater molecular weight can lower the volatility of the molecule, thereby potentially changing a top note to a middle note, or a middle note to a bass note.

Citral is one example of a known aromachemical that can be modified as described above, although it is immediately applicable to any other odorants possessing the same structural features, namely a C═C double bond. Citral can be cyclopropanated in one or both of the positions, corresponding to the two double bonds of the molecule. Cyclopropanation of the double bond alpha to the carbonyl provides four possible diastereomers, (R,R), (R,S), (S,R) and (S,S). Cyclopropanation of the double bond of the isoprene unit provides two stereoisomers for each of the E and Z isomers of the double bond alpha to the carbonyl, for a total of four isomers. Dicyclopropanation provides eight steriosomeric forms, including all four diastereomers from the cyclopropanation of the double bond alpha to the carbonyl coupled with the R stereoisomer from the cyclopropanation of the double bond in the isoprene unit, and all four diastereomers coupled with the S stereoisomer. The double bond in the isoprene unit of citronnelal can also be cyclopropanated to yield two stereoisomers.

The cyclopropanation reaction can be performed to exhaustion, providing the dicyclopropanated product, or can be performed stepwise, yielding a mixture of mono and di-cyclopropanated products. The mono- and di-cyclopropanated products can be separated on the basis of different physical and chemical properties. Diastereomeric forms can be separated on the basis of different properties as well, such as different boiling points and/or crystallization conditions, as is known in the art. Stereoisomers can be isolated using known techniques, such as column chromatography using a chiral solid phase, enzymatic degradation, and reversible formation of diastereomers and separation of the diastereomeric forms, as is known in the art. The presence of the aldehyde functionality permits the rapid and reversible formation of diastereomers by reaction with chiral alcohols to form hemiacetals or acetals, which can be hydrolyzed to reform the aldehyde functionality. Suitable chiral alcohols are well known to those of skill in the art. Accordingly, should it be desired to isolate particular stereoisomers or diastereomers, it would be routine in the art to do so.

While the disclosure is not limited to the following drawing, all sixteen stereoisomeric forms of mono- and di-cyclopropropanated citral derivatives, and both stereoisomeric forms of cyclopropanated citronellal, are shown below. The replacement of double bonds with cyclopropane rings in other compounds will likewise often result in the formation of stereoisomers and/or diastereomers, and individual stereoisomers and/or diastereomers can similarly be isolated using conventional separation techniques. Such stereoisomers and/or diastereomers are intended to be within the scope of the invention described herein.

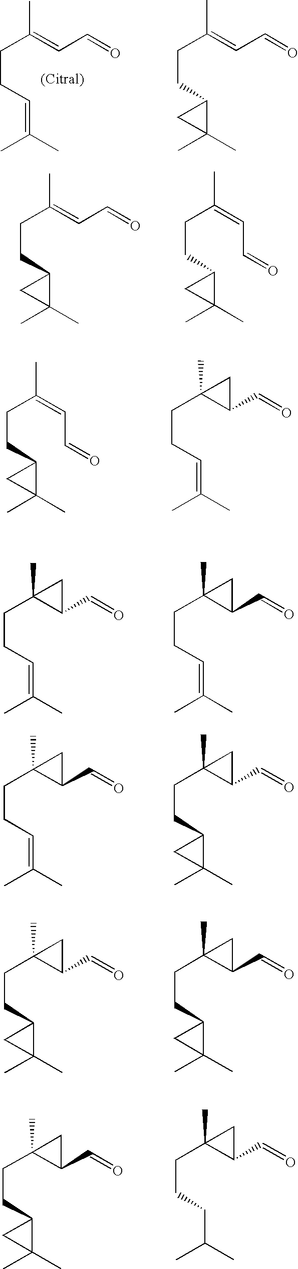

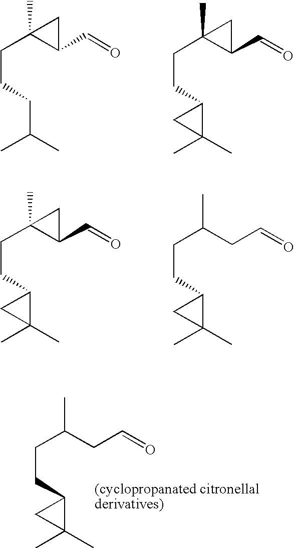

The cyclopropane rings can include a CH₂ moiety, or can be substituted with one or two methyl groups. The methyl or dimethyl analogues have a vibrational spectra that more closely matches citral than the unsubstituted cyclopropane derivatives, and has a sweeter smell than the unsubstituted cyclopropyl derivatives. Further, when aromachemicals include an isoprenyl moiety, it can be replaced with a cyclopropanated version (a dimethyl cyclopropane) where the double bond is replaced with a cyclopropane ring. The cyclopropane ring can further optionally be substituted with additional alkyl groups, but preferably maintains the dimethyl substitution from the original isoprene unit.

The synthesis of methyl, dimethyl or unsubstituted cyclopropane derivatives is well known to those of skill in the art, and involves, for example, bromoform reaction to form the dibromocyclopropane derivative, followed by stoichiometric reaction with methyl lithium. The aldehyde group is typically protected as an acetal during the reaction, and deprotected as desired after the reactions take place. In one embodiment, however, the acetals (for example, dimethyl, diethyl, or ethylene glycol) are not deprotected to the aldehyde, such that the flavoring or fragrance includes a portion or entirely the acetals. The acetals can then slowly hydrolyze over time, releasing the lemon scent/flavoring. Alternatively, derivatives including one or two cyclopropane rings can also include a nitrile or methyl ether group as a replacement for the aldehyde group.

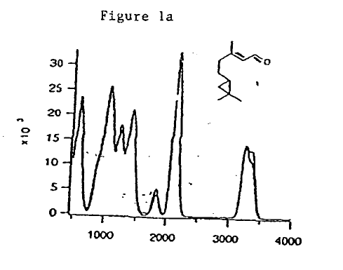

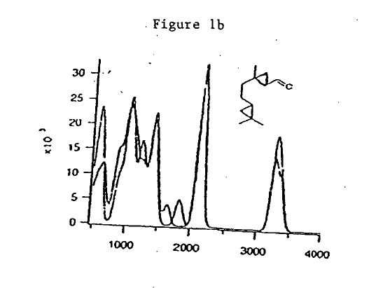

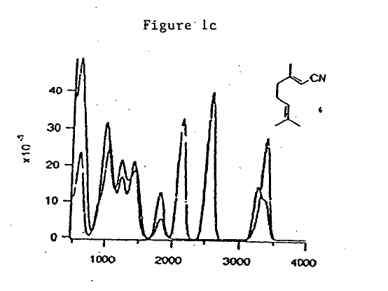

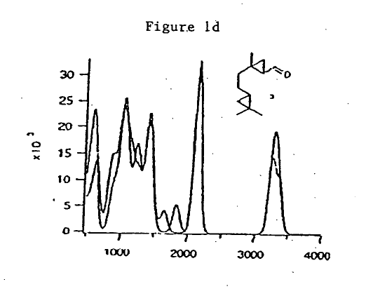

These simple procedures yield derivatives with odor profiles close to the aromachemicals or individual “parent” compounds themselves. Also, the calculated vibrational spectra of the cyclopropyl analogues of citral closely match those of citral, as shown in FIGS. 1A-F. Further, by replacing the double bonds, the derivatives often have greater potency and far greater acid and bleach stability since the unstable feature, namely the double bond, has been removed.

The same applies to epoxide (OX) and thiirane (TH) rings. Not counting mixed C═C double bond replacements and stereoisomers, this generates 9 possible molecules from citral alone, all readily accessible in one or two step syntheses from citral by processes well known in the art, such as:

Cyclopropanyl replacement: Simmons-Smith cyclopropanation of the aldehyde or corresponding alcohol, followed by periodinane oxidation for the latter to give the aldehyde (Vogel's textbook of practical organic chemistry 5th edition (1989) pp 1106-1108).

Oxiranyl replacement: m-chloroperbenzoic acid epoxidation (Ibid, pp 1127-1129)

Thiiranyl replacement: bromination of double bond on Amberlite, followed by S′-substitution in sodium sulfides (Choi, J. et al. (1995) Bull. Korean. Chem. Soc., 16, 189-190 Convenient Synthesis of Symmetrical Sulfides from Alkyl Halides and Epoxides).

In another embodiment, the aromachemicals include an isobutenyl group, and the group is replaced with a phenyl group or a dimethyl cyclopropyl group, particularly where the dimethyl substitution mirrors that in the original isobutenyl group.

Aldehyde Replacement with Nitrile, Methyl Ester, or Acetal Groups

Many aromachemicals also include aldehyde groups. Odotopes of these aromachemicals can be prepared by replacing the aldehyde group with a nitrile, methyl ester or acetal group. The conversion of an aldehyde group to a nitrile group is well known in the art, and described, for example, in U.S. Pat. No. 5,892,092. The '092 patent teaches a process for forming nitrites from aldehydes.

Acetal formation is well known to those of skill in the art, and generally involves reacting an aldehyde with an alcohol in the presence of an acid catalyst. The acetal is formed with loss of water. In use, when the acetal is present in an aqueous environment, the acetal can revert to the aldehyde, thereby providing a time-release form of the odorant.

Aromachemicals including an aldehyde that further include one or more a double bonds can be converted to a thioether, (unsubstituted, methyl or dimethyl) cyclopropyl, oxirane or thiirane, derivative that also include a nitrile, methyl ether or acetal group in place of the aldehyde.

This method is immediately applicable to several other classes of odorants in order to increase their potencies. The following examples each denote a class of odorant, not a single molecule.

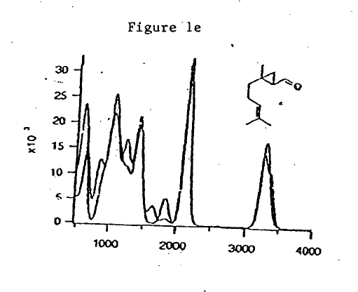

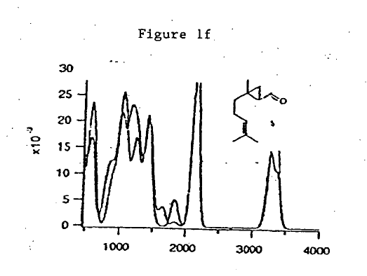

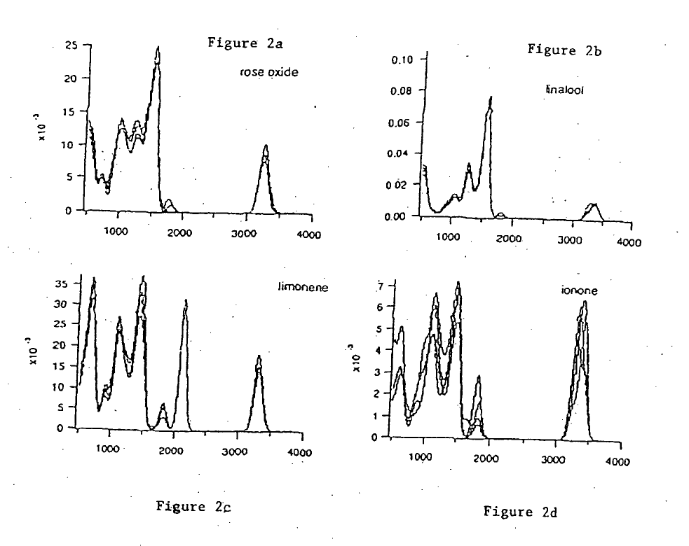

In each case the C═C double bond(s) can be substituted with CP, OX or TH to yield stronger odorants with similar odor profiles. Rose oxide is a floral, ionone a woody violets, damascone a fruity rose, sandanol a sandalwood, limonene a woody citrus, velvione a musk, linalool a floral-woody and ethyl citronellyl oxalate a musk. FIGS. 2A-D represent graphs for four of these compounds with CP, OX and TH substitutions. In each case, the CP, OX and TH substitution has only aminor effect on spectrum and therefore on odor character.

Benzene—Thiophene Replacement

An additional odotopic replacement is a benzene ring for a thiophene ring (where the thiophene ring can optionally be substituted at the 2 and/or 3 positions with a C₁₋₅ alkyl group). For example, when the phenyl rings in lilial, cyclamenaldehyde and bourgeonal are replaced with thiophene, not only do the vibrational spectra overlap and the novel derivatives have the same odor characteristics, but also the intensity of the odor is enhanced. Each of these compounds further includes an aldehyde group that can additionally be replaced with methyl ether, acetal, nitrile, methyl ketone, isocyanate, acetylene, oxime, or ester functionality. Similarly, compounds that include any of the groups in this list can be replaced with another member of this list using known chemistry. Synthetic methods for replacing a phenyl ring in a molecule with a thiophene molecule are well known to those of skill in the art.

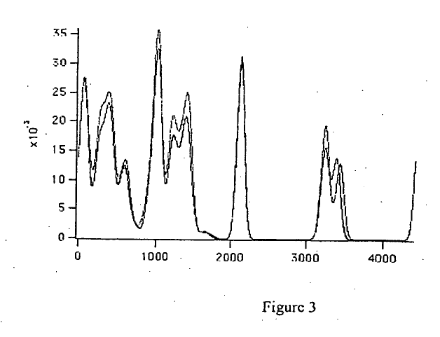

The parent compounds (lilial®, cyclamenaldehyde and bourgeonal®) and the novel compounds including the thiophene ring substitution, are shown below. The vibrational spectra of lilial® and the corresponding molecule where the benzene ring has been replaced with a thiophene ring, is shown in FIG. 3.

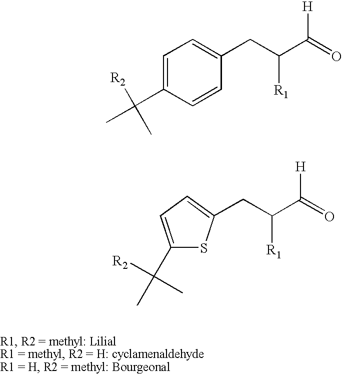

Methods of Enhancing Odorant Stability

Using the information provided herein regarding isodonic molecules, one can evaluate a “parent” odorant molecule and identify portions of the molecule that result in instability in a given environment. Then, using the isodonic replacements described herein, or others that result in a substantially similar vibrational spectra of the odorant derivative relative to the parent odorant, one can devise a derivative that is more stable in the proposed environment of use. For example, if a compound is to be subjected to a strongly acidic or basic environment, alpha-beta unsaturation, carbon-carbon double bonds, and other functional groups can be replaced with isodonic replacements that are less reactive, for example, a cyclopropane ring can be used to replace a carbon-carbon double bond. Those of skill in the art can readily determine which functional groups present in a molecule give rise to instability in a particular environment of use, and which isodonic replacements would improve stability.

Methods of Enhancing Odorant Intensity

Using the information provided herein regarding isodonic molecules, one can evaluate a “parent” odorant molecule and identify portions of the molecule that bind zinc ions. Then, using the isodonic replacements described herein, or others that result in a substantially similar vibrational spectra of the odorant derivative relative to the parent odorant, one can devise a derivative that has an improved ability to bind zinc ions. For example, for compounds that possess carbon-carbon double bonds, replacement of one or more of those carbon-carbon double bonds with isodonic replacements with higher zinc-binding affinity, such as cyclopropane, thiirane, oxirane, or thioether linkages provides isodonic molecules with improved odorant intensity. Those of skill in the art can readily determine which functional groups present in a molecule bind to zinc ions, and which isodonic replacements would improve stability.

Aromachemicals

The technology described herein has particular application to essential oils, including lyral, hydroxycitronellal and alcohols related to citral, including citronellal, geraniol, nerol and the like.

There are several essential oils with aldehyde groups that can be derivatized with acetal, methyl ether or nitrile groups using the chemistry described herein. These include, but are not limited to, angelica, aleprine, alpha,beta-apocitronellal, bergamotene, pyroterebine, campholene, citronellal, citral, chrysantheme, cyclocitral, cyclolavandulal, faranal, farnesal, isolauranal, ikema, myrthenal, phellandrine, safranal oxime and sorbinal oxime. Specific examples include angelica, bergamotene, cyclolavandulal, citral, famesal, ikema, isolauranal, phellandrine oxime and sorbinal oxime.

There are several essential oils (several of which are listed above) that include olefinic groups that can be derivatized by forming an ene-thioether, ene-cyclopropane, ene-oxirane and/or ene-thiirane replacement. The following are specific odorants that can be modified as described herein, wherein the double bonds can be converted to (unsubstituted, methyl or dimethyl) cyclopropyl derivatives, and/or the aldehydes (where present) converted to acetals, methyl ethers or nitriles without significantly altering the odor profile.
Amyl cinnamal (also known as 2-benzylidineheptanal and alpha-amyl cinnamic aldehyde)
Amyl cinnamyl alcohol (also known as 2-pentyl-3-phenylprop-2-ene-1-ol and alpha-amyl cinnamic alcohol)
cinnamyl alcohol (also known as cinnamic alcohol)
cinnamal (also known as 3-phenyl-2-propenal and cinnamic aldehyde)
citral (also known as 3,7-dimethyl-2,6-octadiene-1-al, mix of cis and trans isomers)
coumarin (also known as 1-benzopyran-2-one or cis-o-coumarinic acid lactone)
eugenol
geraniol
hydroxycitronellal (also known as 7-hydroxycitronellal or laurine)
lyral (also known as hydroxymethyl-pentylcyclo-hexenecarboxaldehyde and 4,(4-hydroxy-4-methylpentyl)cyclohex-3-enecarbaldehyde
isoeugenol
benzoyl cinnamate (INCI), (also known as benzyl 3-phenyl-2-propenoate or cinnamein)
citronellol (also known as 3,7-dimethyl-6-octenol)
farnesol (also known as 3,7,11-trimethyldodeca-2,6,10-trienol
hexyl cinnamaldehyde (also known as alpha-hexyl cinnamaldehyde)
lilial (also known as lilestral, 2-(4-tert-butylbenzyl)proprionaldehyde, 4-(1,1-dimethylethyl)-alpha-methylbenzenepropanal, p-tert-butyl-alpha-methylhydrocinnamaldehyde)
d-limonene (also known as (R)-p-mentha-1,8-diene
linalool, and
gamma-methyl ionone ((also known as 3-methyl-4-(2,6,6-trimethyl-2-cyclohexen-1-yl)-3-butene-2-one.

Additionally, the compounds can be selected from anethole, anise oil, caraway oil, cardamom oil, carvone, coriander oil, eriodictyon, ethyl vanillin, fennel oil, glycyrrhiza, lavender oil, lemon oil, menthol, nutmeg oil, orange flower oil, peppermint, rosemary oil, rose oil, spearmint oil, thyme oil, tolu balsam and vanillin.

Additional examples include angelica, bergamotene, cyclolavandulal, citral, famesal, ikema, isolauranal, phellandrine oxime and sorbinal oxime. In particular, citral oxime can be converted to geranonitrile.

Combinatorial Libraries

The methods described herein can be used to design odorant libraries. For example, the libraries can include plurality of odorants and isodonic analogs of one or more of the odorants, or can simply include the analogs themselves, where isodonics are defined as compounds with at least about 85 percent homology between the tunneling vibrational spectra of the odorant and the odotope. The odorant library can also include blends of odorants and/or odotopes.

In one embodiment, at least one of the odorants comprises an olefinic group, and at least one of the odotopes comprises a cyclopropane, oxirane or thiirane group, wherein the cyclopropane can optionally be substituted with one or two methyl groups, or the double bond is replaced with a thioether linkage. In another embodiment, at least one of the odorants comprises a phenyl group, and at least one of the odotopes comprises an isoprene group. In a third embodiment, at least one of the odorants comprises an aldehyde group, and at least one of the odotopes comprises a nitrile, acetal, or methyl ether group. In a fourth embodiment, at least one of the odorants comprises a phenyl group, and at least one of the odotopes comprises a thiophene group.

The odorant library can include one or more odorants selected from the group consisting of insect pheromones, animal pheromones, such as musks, human pheromones, perfumes, flavorings, and odorants derived from aromachemicals.

Ideally, to facilitate rapid screening of the odorant libraries, the odorants are arranged in a matrix-type arrangement of related odors. However, the odorants and odotopes can have substantially different odor intensity, even within a given odorant matrix.

The odorant libraries can be prepared, for example, by obtaining a first library of odorants, and synthesizing odotopes of the first library of odorants by performing one or more of the above-mentioned isodonic replacements on the odorants to prepare odotopes.

The combinatorial libraries can be screened to identify compounds with desired odor characteristics, intensity and/or stability. The screening methods generally involve obtaining a library comprising odorants and isodonics and screening the library for odorants and/or isodonics with desired odor characteristics, odorant intensity and/or odorant stability.

The compounds can be screened for odorant character by comparing the vibrational spectra of the odorants and odotopes with a desired vibrational spectra. Desired odorants or isodonics can be identified by the presence of and/or intensity of certain absorption bands. Desired isodonic compounds can be identified, for example, when their vibrational spectra have at least about 85% overlap with the desired vibrational spectra, at least with respect to certain key absorption bands. This method can be effective in an iterative process, wherein putative compounds are prepared, compounds with desired odor characteristics identified in a first iterative step, and compounds with desired odorant intensity and/or stability identified in subsequent iterative steps. However, the method can also be carried out in a step-wise fashion, wherein all compounds are screened for desired odorant character and also for additional desired physical and/or chemical properties.

After the first iterative step, lead isodonic molecules can be screened for their zinc-binding affinity as described above. This two step process permits one to not only identify molecules with similar odorant character, but also with improved odor intensity. Alternatively, after the first iterative step, lead isodonic molecules can be screened for their stability in a given environment. This can be done in a high throughput manner, for example, by preparing a plurality of tubes (in a multi-tube array) or wells (in a multi-well plate) with a solution approximating a desired environment. Individual compounds to be evaluated can be placed in each tube or well, and the stability of the compounds evaluated over time. The stability can be evaluated, for example, by comparing initial spectra with spectra obtained at different time intervals. In another embodiment, stability is evaluated by taking a bas chromatograph (GC) or high performance liquid chromatograph (HPLC), ideally with an internal standard that is stable under the conditions of use, and following the loss of the compound over time. This two step process permits one to not only identify molecules with similar odorant character, but also with improved stability.

Additional desired properties that can be evaluated include log P values and molecular weight, surface area, volume and ellipticity.

Improved Articles of Manufacture

The combinatorial libraries, and methods for evaluating the libraries, can be used in the perfume industry, beverage industry and the like to identify desirable odorants in a rapid fashion, and tailor odorants to particular environments of use. Further, by identifying odorants with improved intensity, less odorant can be employed to provide articles of manufacture with a desired odor, which can result in significant cost savings.

Examples of suitable articles of manufacture include candles, air fresheners, perfumes, disinfectant compositions, hypochlorite (bleach) compositions, beverages such as beer and soda, denture cleanser tablets and flavored orally-delivered products such as lozenges, candies, and the like.

Having hereby disclosed the subject matter of the present invention, it should be apparent that many modifications, substitutions, and variations of the present invention are possible in light thereof. It is to be understood that the present invention can be practiced other than as specifically described. Such modifications, substitutions and variations are intended to be within the scope of the present application.

1. A method for predicting odorant character comprising:
a) calculating the vibrational frequencies and atom displacements of a putative molecule,
b) calculating the electrostatic charge distribution on the constituent atoms of the putative molecule, and
c) calculating the intensity of each vibrational mode using the information in steps a and b and an algorithm that calculates intensity of vibrational modes as detected or perceived by a solid state electron tunneling spectrometer.

2. The method of claim 1, further comprising smoothing the information into the form of a spectra.

3. The method of claim 1, wherein the method is performed on a plurality of compounds.

4. The method of claim 3, wherein the data for two or more compounds from the plurality of compounds is compared.

5. The method of claim 4, wherein the comparison is used to identify two or more compounds with essentially the same odor character.

6. The method of claim 5, where the spectral information obtained on the two or more compounds overlap by 85% or more.

7. The method of claim 4, wherein the information is mapped in an “odorant map” by a method comprising:
a) breaking all of the spectra up into “n” fragments,
b) scaling the peaks of one compound relative to another compound such that at least one peak is the same height as another,
c) taking the remaining “n-I” peaks and generating a plot with the coordinates of those peaks, and
d) optionally, comparing the location on the odorant map of the various compounds. 8.

8. The method of claim 7, wherein the data is plotted in a three dimensional space, using x, y, and z coordinates, and wherein n is 4 and (n−1) is three, representing the three portions of the spectra whose amplitude can be mapped in x, y and z coordinates.

9. A virtual combinatorial odorant library, wherein the compounds in the virtual library are subjected to the method of claim 1 to determine their odorant character, and wherein the library identifies the compounds both by structure and odorant character.

10. A method of identifying an odorant with an odor essentially the same as the odor of a first compound comprising:
a) generating a virtual combinatorial library including the first compound and one or more additional compounds with odotopic replacements for functional groups on the first compound,
b) calculating the tunneling vibrational spectra of the compounds using the method of claim 1, and
c) comparing the spectra of the compounds to identify odorants with essentially the same odorant character.

11. A combinatorial odorant library comprising one or more odorants and compounds with odotopic replacements for one or more functional groups on the odorants.

12. The odorant library of claim 11, wherein at least one of the odorants comprises an olefinic group, and at least one of the odotopes comprises a cyclopropane, epoxide, oxirane or thiirane group, wherein the cyclopropane can optionally be substituted with one or two methyl groups.

13. The odorant library of claim 11, wherein at least one of the odorants comprises a phenyl group, and at least one of the odotopes comprises an isoprene group.

14. The odorant library of claim 11, wherein at least one of the odorants comprises an aldehyde group, and at least one of the odotopes comprises a nitrile, acetal, or methyl ether group.

15. The odorant library of claim 11, wherein at least one of the odorants comprises a phenyl group, and at least one of the odotopes comprises a thiophene group.

16. The odorant library of claim 11, wherein the odorants comprise one or more odorants selected from the group consisting of insect pheromones, animal pheromones, human pheromones, perfumes, flavorings, and odorants derived from aromachemicals.

17. The odorant library of claim 16, wherein the animal pheremones comprise musks.

18. The odorant library of claim 16, wherein the odorants are arranged in a matrix-type arrangement of related odors.

19. A method for identifying compounds with similar odorant character to a known odorant, comprising:
a) preparing a combinatorial library of compounds with odotopic replacements for one or more functional groups on the odorants, and
b) identifying compounds with the odotopic replacements that have similar odorant character to the known odorants.

20. The method of claim 19, further comprising subjecting the compounds with the odotopic replacements identified in step b) to one or more additional analyses to identify compounds with an additional desired physical and/or chemical property.

21. The method of claim 20, wherein the additional property is selected from the group consisting of odor intensity, stability in a desired environment, and log P values.

22. A method for, given an odorant with a phenyl or an isoprene unit, preparing an odorant with similar odorant character, comprising:
replacing the isoprene unit or phenyl ring with an isoprene unit wherein the double bond is replaced with a three membered ring including the two atoms present in the double bond and oxygen, sulfur or C(R)₂,
wherein R is independently selected from the group consisting of H, C₁₋₅ alkyl, and C₁₋₅ substituted alkyl, and
wherein the substituents on the substituted alkyl are selected from the group consisting of halo, hydroxy, thiol, thioether, amine, carboxylic acid, ester, nitro, cyano, isocyano, sulfonic acid, urea and thiourea.

23. A method for, given an odorant with a phenyl or an isoprene unit, preparing an odorant with similar odorant character, comprising:
replacing the isoprene unit or phenyl ring with a thiophene ring,
wherein the thiophene is optionally substituted with one or more C₁₋₅ alkyl or C₁₋₅ substituted alkyl groups, and
wherein the substituents on the substituted alkyl are selected from the group consisting of halo, hydroxy, thiol, thioether, amine, carboxylic acid, ester, nitro, cyano, isocyano, sulfonic acid, urea and thiourea.

24. A method for identifying odorants with similar odor characteristics to existing odorants comprising:
a) calculating the tunneling vibrational spectra of a first odorant,
b) identifying a second odorant with a tunneling vibrational spectra that substantially overlaps the tunneling vibrational spectra of the first odorant.

25. The method of claim 24, further comprising determining the intensity of the second odorant relative to the first odorant.

26. The method of claim 24, further comprising determining the stability of the second odorant when subjected to various physical and/or chemical environments relative to the first odorant.

27. The method of claim 24, wherein the substantial overlap is defined as at least about 85 percent homology between the tunneling vibrational spectra of the first and second odorant.

28. A method for identifying odorants with similar odor characteristics to existing odorants comprising:
a) obtaining the tunneling vibrational spectra of a first odorant,
b) identifying a second odorant with a tunneling vibrational spectra that substantially overlaps the tunneling vibrational spectra of the first odorant.

29. The method of claim 28, further comprising determining the intensity of the second odorant relative to the first odorant.

30. The method of claim 29, further comprising determining the stability of the second odorant when subjected to various physical and/or chemical environments relative to the first odorant.

31. The method of claim 29, wherein the substantial overlap is defined as at least about 85 percent homology between the tunneling vibrational spectra of the first and second odorant.

32. A method for preparing an odorant library, comprising:
obtaining a first library of odorants, and
synthesizing odotopes of the first library of odorants by performing one or more of the following replacements on an odorant to prepare an odotope:
aldehyde to nitrile,
aldehyde to methyl ether,
aldehyde to acetal,
aldehyde to oxime,
aldehyde to methyl ketone,
aldehyde to ester,
olefin to cyclopropane,
olefin to epoxide,
olefin to thiirane,
phenyl to isoprene,
nitrile to aldehyde,
methyl ether to aldehyde,
acetal to aldehyde,
cyclopropane to olefin,
epoxide to olefin,
thiirane to olefin,
isoprene to phenyl,
isoprene or phenyl to cyclopropanated isoprene,
isoprene or phenyl to thiophene, wherein the thiophene can optionally be substituted with one or more C₁₋₅ alkyl or substituted alkyl groups,
and
wherein the cyclopropane can optionally be substituted with one or two methyl groups.

33. A high throughput method for screening compounds for desired odor characteristics comprising:
obtaining a library comprising odorants and odotopes and
screening the library for odorants and/or odotopes with desired odor characteristics.

34. The method of claim 33, wherein the screening is performed by comparing the vibrational spectra of the odorants and odotopes with a desired vibrational spectra.

35. The method of claim 34, wherein the vibrational spectra of the odorants and odotopes identified as having desired odor characteristics have at least about 85% overlap with the desired vibrational spectra.

36. The method of claim 33, further comprising evaluating odorants and/or odotopes identified by the method to identify those odorants and/or odotopes with desired odor intensity.

37. The method of claim 33, further comprising evaluating odorants and/or odotopes identified by the method to identify those odorants and/or odotopes with one or more desired properties selected from the group consisting of stability to a pre-selected chemical environment, log P values, zinc binding affinity, intensity, and stability in an environment to which the odorants might be exposed.

38. The method of claim 33, wherein the library includes blends of odorants and/or odotopes.

39. The method of claim 33, wherein the library includes odotopes related to odorants by one or more of the following chemical substitutions:
aldehyde to nitrile,
aldehyde to methyl ether,
aldehyde to acetal,
aldehyde to oxime,
aldehyde to methyl ketone,
aldehyde to ester,
olefin to cyclopropane,
olefin to epoxide,
olefin to thiirane,
phenyl to isoprene,
nitrile to aldehyde,
methyl ether to aldehyde,
acetal to aldehyde,
cyclopropane to olefin,
epoxide to olefin,
thiirane to olefin,
isoprene to phenyl,
isoprene or phenyl to cyclopropanated isoprene,
isoprene or phenyl to thiophene, wherein the thiophene can optionally be substituted with one or more C₁₋₅ alkyl or substituted alkyl groups,
and
wherein the cyclopropane can optionally be substituted with one or two methyl groups.
